SEQUENCE LISTING (4 sequences)

SEQ ID NO: 1 - DNA, 7 bp, Homo sapiens
gccccct

SEQ ID NO: 2 - DNA, 6 bp, Homo sapiens
gcccct

SEQ ID NO: 3 - DNA, 14 bp, Artificial Sequence
  discriminating primer
gaaggcccctggac

SEQ ID NO: 4 - DNA, 16 bp, Artificial Sequence
  amplicon primer
gctccgaggacgagac

(19) United States
(12) Patent Application Publication
(10) Pub. No.: US 2005/0136463 A1
(43) Pub. Date: Jun. 23, 2005

(54) Method for isolating nucleic acid using alumina

(76) Inventors: Joon-ho Kim, Seongnam-si (KR); Yoon-kyoung Cho, Suwon-si (KR); Geun-bae Lim, Pohang-si (KR); Soo-hwan Jeong, Suwon-si (KR)

Correspondence Address: CANTOR COLBURN, LLP, 55 GRIFFIN ROAD SOUTH, BLOOMFIELD, CT, 06002, US

(21) Appl. No.: 11/016,447

(22) Filed: Dec. 17, 2004

(30) Foreign Application Priority Data
Dec. 18, 2003 (KR) 2003-93172

(51) Int. Cl. C12Q 1/68; C07H 21/04

(52) U.S. Cl. 435/6; 536/25.4

(57) ABSTRACT

A method for isolating a nucleic acid is provided. The method includes mixing a nucleic acid-containing sample, a salt solution, and alumina; isolating the alumina having the nucleic acid attached thereto from the liquid; washing the alumina having the nucleic acid attached thereto; and eluting the nucleic acid from the alumina.

### BACKGROUND OF THE INVENTION

This application claims the benefit of Korean Patent Application No. 10-2003-0093172, filed on Dec. 18, 2003, in the Korean Intellectual Property Office, the disclosure of which is incorporated herein in its entirety by reference.

1. Field of the Invention

The present invention relates to a method for isolating a nucleic acid using alumina.

2. Description of the Related Art

Methods for isolating nucleic acids using solid phase materials are known in the art. For example, U.S. Pat. No. 5,234,809 discloses a method for isolating a nucleic acid using a solid phase to which the nucleic acid may bind. The method includes mixing a starting material, a chaotropic material, and a nucleic acid-binding solid phase; separating the solid phase with the nucleic acid bound thereto from the liquid; and washing the solid phase nucleic acid complexes. Examples of the chaotropic material include guanidinium thiocyanate (GuSCN), guanidine hydrochloride (GuHCl), sodium iodide (Nal), potassium iodide (KI), sodium thiocyanate (NaSCN), urea, a combination thereof, and so on. Examples of the solid phase include silica particles.

However, the method requires the use of the chaotropic material, and without the chaotropic material, the nucleic acid cannot bind to the solid phase. In addition, the chaotropic material is harmful to the human body and should be handled with care. Further, since the chaotropic material inhibits subsequent processes, such as a polymerase chain reaction (PCR), it must be removed during the isolation or from the nucleic acids after the isolation.

U.S. Pat. No. 6,291,166 discloses a method for archiving nucleic acid using a solid phase matrix. The method includes irreversibly binding single- or multi-stranded nucleic acid contained in an aqueous specimen to a solid phase matrix, wherein the solid phase matrix is defined by specific binding materials characterized by electropositive material rendered hydrophilic, manipulating the solid phase bound nucleic acid, and storing the bound nucleic acid on the solid phase matrix. The solid phase matrix may consist of silicon (Si), boron (B), or aluminum (Al). The electropositive material may be rendered hydrophilic using a basic solution, such as a NaOH solution. In the method, manipulating the nucleic acid, which irreversibly binds to the solid phase, includes an enzyme reaction, hybridization, signal amplification, and target amplification. The target amplification includes PCR, SDA, NASBA, IsoCR, CRCA, Q beta replicase, and branched chain DNA method. Since the nucleic acid irreversibly binds to the solid phase matrix, advantageously the solid phase matrix-nucleic acid complexes can be stored and later subject to a delayed analysis or a repeated analysis. However, in the method, the material having a surface that is positively charged, such as aluminum, should be rendered hydrophilic using a basic substance, such as NaOH. Further, a nucleic acid irreversibly binds to the aluminum which has been rendered hydrophilic, and thus it is impossible to isolate the nucleic acid from the aluminum. Accordingly, an ordinary person in the art understands that aluminum may not be used to isolate nucleic acids.

The present inventors conducted research on a method for isolating a nucleic acid based on the conventional methods and discovered the present invention in which a nucleic acid can reversibly bind to aluminum.

### SUMMARY OF THE INVENTION

The present invention provides a method for efficiently isolating a nucleic acid using alumina.

According to an aspect of the present invention, there is provided a method for isolating a nucleic acid using alumina, comprising: mixing a nucleic acid-containing sample, a salt solution, and alumina; isolating the alumina having the nucleic acid attached thereto from the liquid; washing the alumina having the nucleic acid attached thereto; and eluting the nucleic acid from the alumina.

### BRIEF DESCRIPTION OF THE DRAWINGS

The above and other features and advantages of the present invention will become more apparent by describing in detail exemplary embodiments thereof with reference to the attached drawings in which:

FIG. 1 is a view illustrating results of gel electrophoresis analysis for PCR products obtained by performing a PCR in which a product isolated by using alumina and a chaotropic material was used as a template;

FIG. 2 is a view illustrating a result of agarose gel electrophoresis for a product isolated by a method according to an embodiment of the present invention;

FIG. 3 is a view illustrating a result of gel electrophoresis analysis for a PCR product obtained by performing a PCR in which a product isolated by a method according to an embodiment of the present invention was used as a template;

FIG. 4 is a view illustrating a result of a PCR in which a HBV plasmid DNA isolated from the HBV plasmid DNA mixture solution with or without 20% PEG by a method according to an embodiment of the present invention was used as a template;

FIG. 5 is a view illustrating a result of gel electrophoresis analysis for a PCR product obtained by performing a PCR in which an DNA isolated by filtering a DNA containing sample through a flow-through type alumina was used as a template; and

FIG. 6 is a view of illustrating a result of determining the concentration of a PCR product based on a band observed through the gel electrophoresis analysis in FIG. 5.

### DETAILED DESCRIPTION OF THE INVENTION

According to an embodiment of the present invention, there is provided a method for isolating a nucleic acid using alumina, comprising: mixing a nucleic acid-containing sample, a salt solution, and alumina; isolating the alumina having the nucleic acid attached thereto from the liquid; washing the alumina having the nucleic acid attached thereto; and eluting the nucleic acid from the alumina.

In an embodiment of the present invention, the nucleic acid-containing sample may be a biological material containing a nucleic acid. Examples of the biological material include whole blood, serum, buffy coat, urine, feces, cerebrospinal fluid, sperm, saliva, tissues, cell cultures, and so on. Also, the nucleic acid-containing sample may be a non-biological material containing a nucleic acid. For the biological sample, if a barrier, such as cell wall, cell membrane, or envelop, prevents direct contact of the nucleic acid with the alumina, a pretreatment can be performed with a substance that can destroy the barrier, such as a detergent or an organic solvent. For example, a cell may be broken up using NaOH and made neutral, and then the solvent may be replaced by a salt (such as NaCl) solution used in an embodiment of the present invention, for a subsequent purification.

As used herein, the term salt solution refers to a salt solution that contributes to the stabilization of the nucleic acids. Thus, the salt solution does not contain a chaotropic salt therein. The salt solution may be a solution containing at least one salt selected from the group of consisting of NaCl, MgCl₂, KCl, CaCl₂, and a combination thereof. The salt may be present in the salt solution at a concentration of O.5to5 M.

As used herein, the term alumina refers to an aluminum oxide. The alumina may be in different forms, such as Al₂O₃ crystals and powders, membranes, plates, beads, and so on. The alumina may be a porous alumina.

In an embodiment of the present invention, the alumina having the nucleic acid attached thereto may be isolated from the liquid, for example, by performing a centrifugation or directly removing the alumina from the liquid.

In an embodiment of the present invention, washing the alumina having the nucleic acid attached thereto may be carried out using a wash buffer containing ethanol and EDTA.

In an embodiment of the present invention, eluting the nucleic acid from the alumina may be carried out using at least one selected from the group consisting of water, Tris buffer, and PBS.

The present invention will be described in more detail by presenting examples. These examples are for illustrative purpose and are not intended to limit the scope of the present invention.

### EXAMPLES

### Example 1

Isolation of HBV Plasmid DNA Using an Alumina and a Chaotropic Material

In Example 1, a plasmid DNA was attached to a surface of alumina using a chaotropic material for comparative purpose and isolated by elution.

An anodized aluminium oxide (AAO) (available from Whatman, trade name: anodisc™) was used as alumina in Example 1. The alumina had a pore size of 200 nm and a thickness of no more than 60 μm. HBV plasmid DNA (about 7.3 kb, ATCC No. 45020D) was used as the plasmid DNA. The isolation procedure was as follows.

1. HBV plasmid DNA was dissolved in distilled water at a concentration of 1 ng/μl.

2. 200 μl (200 ng) of the HBV plasmid DNA solution was mixed with 800 μl of Binding Buffer (5M GuSCN, Triton X-100, Tris-HCl, pH 7.4 (available from Nuclisens)).

3. An anotop filter equipped with an alumina filter therein was connected to a tygon tube and a peristaltic pump. A container having 200 μl a of the DNA mixture obtained above was connected to an inlet of the peristaltic pump. Then, the peristaltic pump was operated so that a constant flow of DNA passed through the alumina filter.

4. After eluting with 1 ml of a wash buffer I (5M GuSCN, Triton X-100, Tris-HCl), a container having 1 ml of a wash buffer II (70% ethanol+10 mM EDTA solution) was connected to an inlet of the peristaltic pump. Then, the peristaltic pump was operated so that a constant flow of wash buffer II passed through the alumina filter.

5. A container having an elution solution was connected to an inlet of the peristaltic pump, the elution solution being, respectively, A: Tris-HCl, pH 7.4 (available from Nuclisens), B: Tris-HCl, pH 8.8, and C: NaOH, pH 10. Then, the peristaltic pump was operated so that a constant flow of the solution passed through the alumina filter. 100 μl of the elution solution thus obtained was collected.

6. PCR was performed using 100 μl of the each elution solution as a template and using oligonucleotides having SEQ ID NOs. 1 and 2 (the size of the amplified product was about 100 bp) as a primer. Cycle conditions were 40 cycles at 95° C. for 20 sec, at 58° C. for 30 sec, and at 72° C. for 40 sec using an MJ Research PTC-100 apparatus.

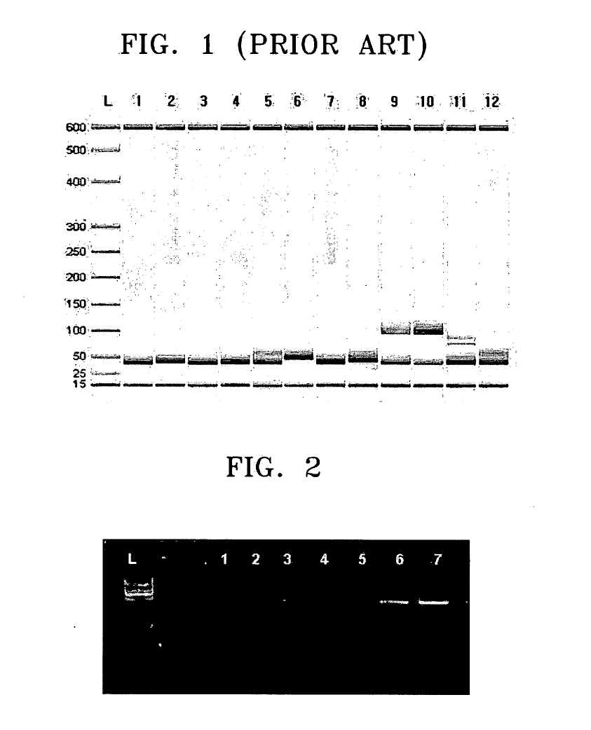

The PCR products were respectively subject to an agarose gel electrophoresis, and their concentrations were determined. The results are shown in FIG. 1. Referring to FIG. 1, lanes 1 to 3 indicate the results using NaOH, pH 10, as an elution solution; lanes 4 to 8 indicate the results using Tris-HCl, pH 8, as an elution solution; and lanes 9 to 11 indicate the results using Tris-HCl (available from Nuclisense), pH 7.4, as an elution solution. As illustrated in FIG. 1, when the binding buffer containing a chaotropic material is used, the nucleic acids may not be efficiently isolated. That is to say, in Example 1, the nucleic acids were attached to a week electropositive surface of the alumina using the binding buffer (pH 7.4) containing the chaotropic material, and then the surface charge of the alumina was made electronegative by using the elution solution with a higher pH during the elution. However, the nucleic acids were not easily eluted.

### Example 2

Isolation of pBR322 Plasmid Using a Salt

In Example 2, pBR322 plasmid DNA was attached to a surface of alumina using a solution containing pBR322 plasmid DNA as a sample and isolated by elution.

First, an anodized aluminium oxide (AAO) (available from Whatman, trade name: anodisc™) was used as an alumina. The alumina had a pore size of 200 nm and a thickness of no more than 60 μm. pBR322 plasmid DNA (about 4.2 kb, available from Promega) was used as the plasmid DNA. The isolation procedure of the pBR322 plasmid DNA was as follows.

1. pBR322 plasmid DNA was dissolved in distilled water at a concentration of 10 ng/μl.

2. 100 μl (1 μg) of the pBR322 plasmid DNA solution was mixed with 100 μl of a 2.5 M NaCl solution.

3. 180 μl of the mixture was injected into a polymer chamber of dimensions 1.6 mm×1.6 mm×0.4 mm and containing an AAO membrane (diameter 1 cm, pore size 200 nm), the polymer chamber having an inlet and an outlet for fluids.

4. After being left at room temperature for 5 minutes, the sample solution was removed from the AAO with a pipette.

5. The chamber was washed by injecting 180 μl of a washing buffer (70% ethanol+10 mM EDTA solution) into the chamber, and the washing was repeated three times.

6. The washing buffer was removed from the chamber and distilled water was injected into the chamber to elute the DNA from the alumina membrane.

7. The eluted DNA was confirmed by an agarose gel electrophoresis.

The results of the agarose gel electrophoresis are shown in FIG. 2.

Referring to FIG. 2, lane 1 indicates the result of a negative control, lane 3 indicates the result of isolating of a nucleic acid according to an embodiment of the present invention, and lanes 5 to 7 indicate the results of 25 ng, 50 ng, and 100 ng of pBR322 plasmid DNAs that were not subject to the isolation procedure.

As illustrated in FIG. 2, pBR322 plasmid DNA can be isolated using alumina and NaCl.

### Example 3

Isolation of HBV Plasmid DNA and its Use as a Template in PCR

In Example 3, HBV plasmid DNA was attached to a surface of alumina using a solution containing HBV plasmid DNA as a sample and isolated by elution. Also, it was confirmed whether the isolated DNA may be directly used as a template or not.

First, an anodized aluminium oxide (AAO) (available from Whatman, trade name: anodisc™) was used as an alumina. The alumina had a pore size of 200 nm and a thickness of no more than 60 μm. HBV plasmid DNA (about 7.3 kb, ATCC No. 45020D) was used as the plasmid DNA. The isolation procedure of the HBV plasmid DNA was as follows.

1. HBV plasmid DNA was dissolved in distilled water at a concentration of 10 ng/μl.

2. 100 μl of the HBV plasmid DNA solution prepared was mixed with 100 μl of a 2.5 M NaCl solution.

3. 180 μl of the DNA containing mixture was injected into a polymer chamber of dimensions 1.6 mm×1.6 mm×0.4 mm and containing an AAO membrane, the polymer chamber having an inlet and an outlet for fluid.

4. The DNA sample was left at room temperature for 5 minutes after injection.

5. The DNA sample solution was removed from the AAO with a micropipette.

6. The chamber was washed by injecting 1 ml of a washing buffer consisting of 70% ethanol+10 mM EDTA solution into the chamber, and the washing was repeated three times.

7. The washing solution was removed from the chamber, and 180 μl of distilled water was injected into the chamber to elute the DNA from the alumina membrane. Then, PCR was performed using 180μl of the eluted DNA solution as a template and using oligonucleotides having SEQ ID NOs. 1 and 2 (the size of the amplified product is about 100 bp) as a primer. Cycle conditions were 40 cycles at 95° C. for 20 sec, at 58° C. for 30 sec, and at 72° C. for 40 sec using an MJ Research PTC-1 00 apparatus.

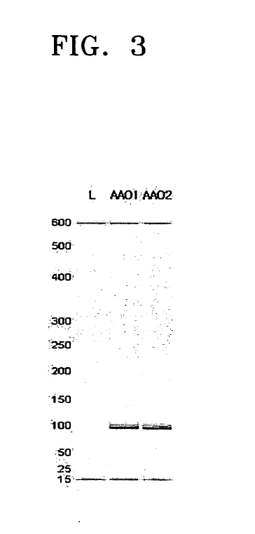

8. After PCR amplification, the concentration and size of the PCR product were confirmed by an electrophoresis apparatus, Agilent 2100 Bioanalyzer (available from Agilent). The results are shown in FIG. 3. In FIG. 3, AAO1 and AAO2 indicate the results of repeated experiments under the same conditions. As illustrated in FIG. 3, according to an embodiment of the present invention, 7.3 kb plasmid DNA was isolated, and the PCR amplification was performed using the isolated plasmid DNA to obtain an amplified PCT product having a size of about 100 bp. Thus, the method according to an embodiment of the present invention can be used to isolate DNA that is useful as a template in PCR.

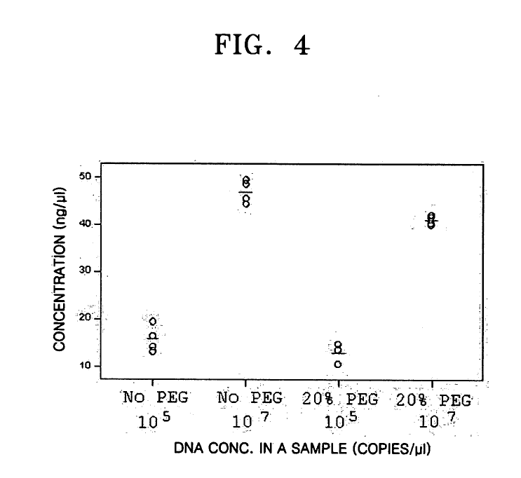

In addition, the above procedure was repeated with the same conditions except that the initial concentration of HBV plasmid DNA was changed to 10⁵ copies/μl and 10⁷ copies/μl, respectively, and that when mixing in the above step 2, 20% PEG was further added. The results are shown in FIG. 4.

### Example 4

### Flow-Through Type Isolation Using an Alumina Membrane

In Example 4, DNA was isolated by passing a DNA-containing sample through an alumina membrane.

Alumina membrane used was an anodized oxide membrane filter (available from Whatman, catalogue No. 6809-1102, trade name: anotop™), which was equipped in a housing made of polypropylene. The alumina had a pore size of 200 nm, a thickness of no more than 60 μm, and a membrane diameter of 10 mm. HBV plasmid DNA (about 7.3 kb, ATCC No. 45020D) was used as the plasmid DNA. A peristaltic pump (available from ISMATIC) was used to pass the DNA-containing sample through an alumina filter. The isolation procedure of the HBV plasmid DNA was as follows.

1. HBV plasmid DNA was dissolved in distilled water at a concentration of 10 ng/μl.

2. 100 μl of the HBV plasmid DNA solution was mixed with 100 μl of a 2.5 M NaCl solution.

3. An anotop filter equipped with an alumina filter therein was connected to a tygon tube and a peristaltic pump. A container having 200 μl of the DNA mixture obtained above was connected to an inlet of the peristaltic pump. Then, the peristaltic pump was operated so that a constant flow of DNA passed through the alumina filter.

4. A container having 1 ml of a wash buffer (70% ethanol+10 mM EDTA solution) was connected to an inlet of the peristaltic pump. Then, the peristaltic pump was operated so that a constant flow of wash buffer passed through the alumina filter.

5. A container having distilled water was connected to an inlet of the peristaltic pump. Then, the peristaltic pump was operated so that a constant flow of the distilled water passed through the alumina filter. 200 μl of the elution solution thus obtained was collected.

6. PCR was performed using 10 μl of the elution solution and using oligonucleotides having SEQ ID NOs. 1 and 2 as a primer. Cycle conditions were 40 cycles at 95° C. for 20 sec, at 58° C. for 30 sec, and at 72° C. for 40 sec using an MJ Research PTC-100 apparatus.

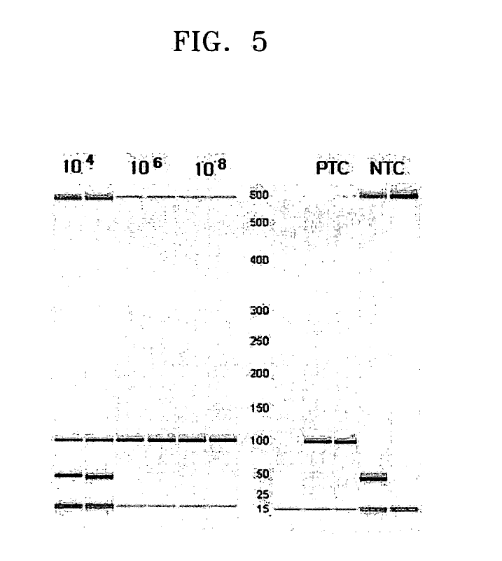

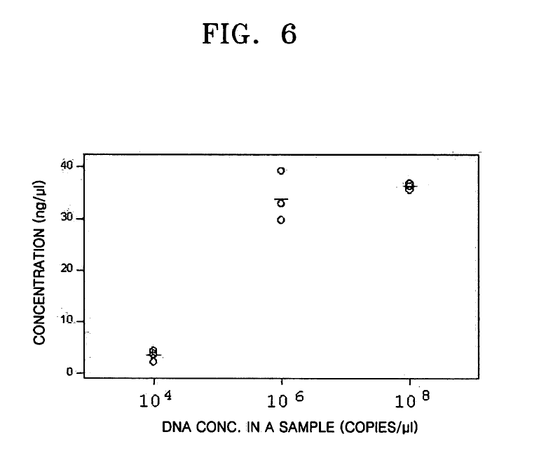

The PCR product was subject to electrophoresis using an Agilent Bioanalyzer, and its concentration was determined. The results are shown in FIGS. 5 and 6. FIG. 5 is a view illustrating a result of gel electrophoresis analysis for the PCR product. As illustrated in FIG. 6, when the initial concentration was 10⁴ copies/μl, 10⁶ copies/μl, or 10⁸ copies/μl, the PCR products were obtained. Referring to FIG. 5, PTC indicates a positive control and NTC indicates a negative control.

The results of Example 4 demonstrate that DNA can be isolated by passing a DNA-containing sample through an alumina filter.

The alumina used in an embodiment of the present invention has a surface that is positively charged, and thus has a high affinity to nucleic acids that are negatively charged. In addition, a porous alumina, of which production is easy, has a large surface area and thus is advantageously used for isolation.

Thus, according to an embodiment of the present invention, the nucleic acid can be efficiently isolated using alumina without a chaotropic material.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method for isolating a nucleic acid using alumina, comprising:
mixing a nucleic acid-containing sample, a salt solution, and alumina;
isolating the alumina having the nucleic acid attached thereto from the liquid;
washing the alumina having the nucleic acid attached thereto; and
eluting the nucleic acid from the alumina.

2. The method of claim 1, wherein the nucleic acid-containing sample is a biological material containing a nucleic acid.

3. The method of claim 1, wherein a salt of the salt solution is at least one selected from the group of consisting of NaCl, MgCl₂, KCl, CaCl₂, and a combination thereof.

4. The method of claim 3, wherein the salt is present in the salt solution at a concentration of 0.5 to 5 M.

5. The method of claim 1, wherein the alumina is in the form of a particle, a membrane, or a plate.

6. The method of claim 1, wherein the alumina is a porous alumina.

7. The method of claim 1, wherein the alumina having the nucleic acid attached thereto is isolated from the liquid by precipitation or centrifugation.

8. The method of claim 1, wherein washing the alumina having the nucleic acid attached thereto is carried out using a wash buffer containing ethanol and EDTA.

9. The method of claim 1, wherein eluting the nucleic acid from the alumina is carried out using at least one selected from the group consisting of water, Tris buffer, and PBS.
